(19) United States
(12) Patent Application Publication
(10) Pub. No.: US 2008/0228044 A1
(43) Pub. Date: Sep. 18, 2008

(54) Individually Activated Sensors for Implantable Sensors

(75) Inventors: Ralph Kurt, Eindhoven (NL); Mark T. Johnson, Veldhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, N.V., Eindhoven (NL)

(21) Appl. No.: 12/089,772

(22) PCT Filed: Oct. 6, 2006
(86) PCT No.: PCT/IB06/53668

§ 371 Date: Apr. 10, 2008

Related U.S. Application Data
(60) Provisional application No. 60/725,221, filed on Oct. 11, 2005.

(51) Int. Cl. A61B 5/07; A61B 5/00

(52) U.S. Cl. 600/300

(57) ABSTRACT

An apparatus for managing and monitoring a sensing device encapsulated in a compartment formed within a medium is disclosed. The medium compartment includes a release mechanism (210) suitable for exposing the sensing device (310). The apparatus comprises an active component (510) connected to the encapsulated sensing device, the active component providing a measurement from the sensing device to a sensing measurement device. In one aspect of the invention a second active device (420) connected to an electrode associated with the release mechanism, the second active component (420) selectively providing an electrical signal to the electrode for activating the release mechanism (210) and exposing the encapsulated sensing device (310). In another aspect of the invention, a plurality of the apparatus disclosed may be incorporated into an array that is electrically connected to a select circuit for providing a voltage to selected ones of the first and second active devices for switching the active devices to a conductive state. A release circuit selectively provides a voltage to selected ones of the second active device (420), wherein the voltage is suitable for operating an associated compartment release mechanism and exposing the associated sensing device.

This invention relates to the field of bio-implantable sensors and more specifically to coupled matrix addressing of implantable sensors for activating and sensing.

Development of bio-implantable sensors provides a significant benefit to people who must continuously monitor their physical condition. For example, diabetes patients typically monitor their glucose levels by using a finger prick and insulin injection procedure. This procedure must be performed several times a day. This procedure is burdensome and problems with existing glucose monitoring technology have resulted in poor compliance with the recommended monitoring guidelines.

Researchers have tried numerous schemes for implantable glucose sensors but have had difficulty keeping them functional once implanted. The body forms scar tissue around foreign material, thus, preventing a sensor from providing accurate readings. However, research of implantable glucose monitoring devices has produced significant advances and the commercialization in such devices. See, for example, “R.F. Service,” Science 297, 962 (2002), and “Continuous Glucose Monitoring: Long-Term Implantable Sensor Approach,” Diabetes Technology & Therapeutics, September 1999, vol. 1, No. 3, pp. 261-266.

However, sensor signals have been found to deteriorate after prolonged implantation due to biofouling, for example. US Published Patent Application Serial No 20050096587, published May 5, 2005, teaches multiple reservoirs to protect and selectively expose sensors or other reservoir contents to reduce the biofouling of individual sensors. This patent application introduces a thermal destruction mechanism by heating the enclosed glucose oxidase to a temperature effective to deactivate the enzyme. This eliminates the possibility of residual peroxide formation and the risk of resulting sensor cross talk. However, this application teaches a complex mechanism for exposing the enclosed sensors.

Hence, there is a need in the industry for a long-term, single, implantable device, suitable for glucose monitoring, to provide on-demand real-time monitored levels and trends.

An apparatus for managing and monitoring a sensing device encapsulated in a compartment formed within a medium is disclosed. The medium compartment includes a release mechanism suitable for exposing the sensing device. The apparatus comprises an active component connected to the encapsulated sensing device, the active component providing a measurement for the sensing device to a sensing measurement device. In one aspect of the invention, a second active component is connected to an electrode associated with the release mechanism, the second active component providing an electrical signal for activating the release mechanism and exposing the encapsulated sensing device. In another aspect of the invention, a plurality of the apparatus disclosed are incorporated into an array and electrically connected to a select circuit for selectively providing a voltage to the active devices suitable for switching the active devices to a conductive state. A release circuit selectively provides a voltage to the second active device, wherein the voltage is suitable for operating an associated compartment release mechanism.

FIG. 1 illustrates a multi-reservoir controlled drug delivery system;

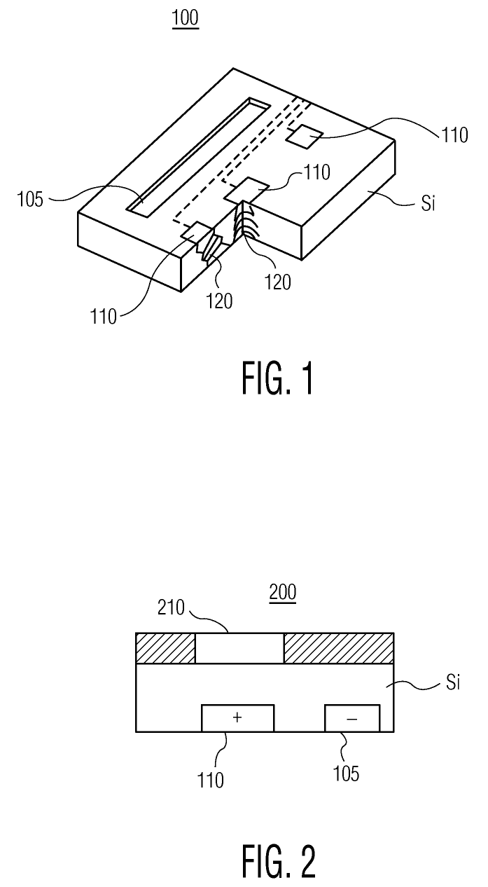

FIG. 2 illustrates a cross-sectional view of an exemplary bio-implantable sensor in accordance with the principles of the invention;

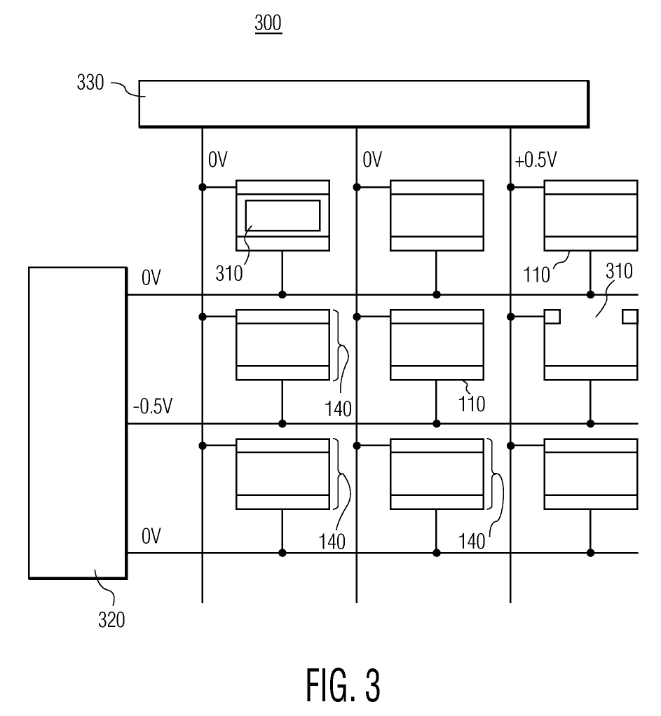

FIG. 3 illustrates a passive control circuit for managing an array of bio-implantable sensors;

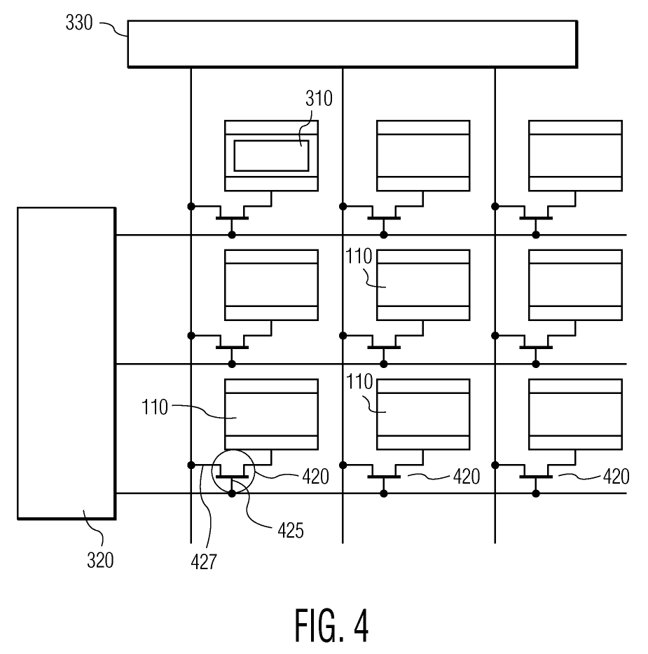

FIG. 4 illustrates an active control circuit for managing an array of bio-implantable sensors;

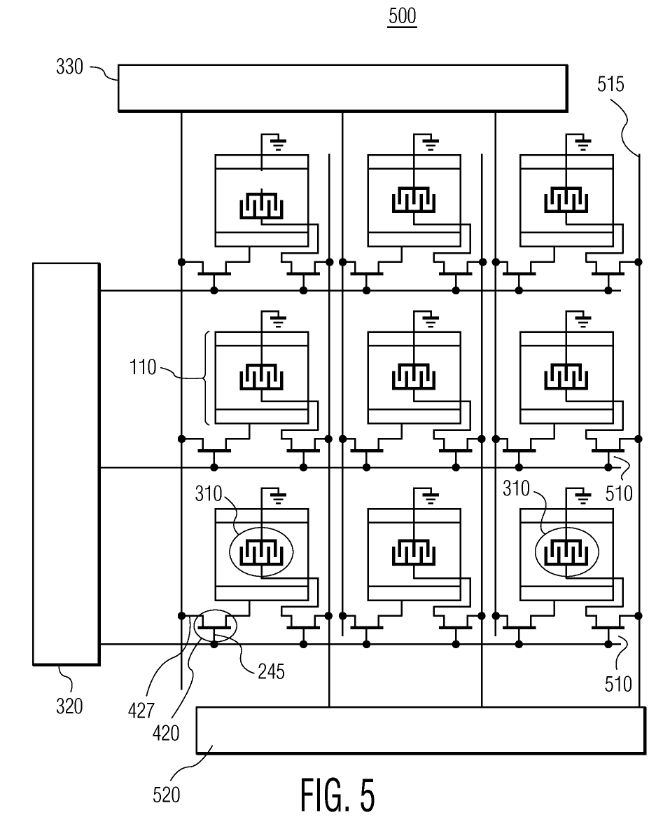

FIG. 5 illustrates an active control circuit for managing and sensing an array of bio-implantable sensors in accordance with the principles of the invention; and

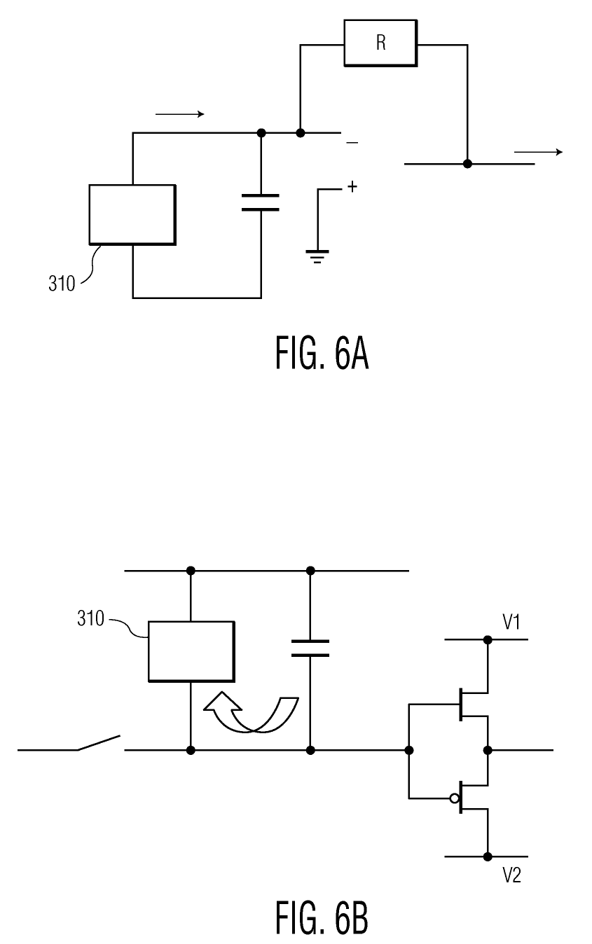

FIGS. 6A and 6B illustrate exemplary amplification circuits for amplifying detected sensor signals.

It is to be understood that these drawings are for purposes of illustrating the concepts of the invention and are not to scale. It will be appreciated that the same reference numerals, possibly supplemented with reference characters where appropriate, have been used throughout to identify corresponding parts.

FIG. 1 illustrates an exemplary multi-reservoir controlled drug delivery system 100 similar to that which is more fully described in “Biocompatibility and Biofouling of MEMS Drug Delivery Device,” Biomaterials 24, p. 1959-1967 (2003). In this illustrated device, a plurality of reservoirs or compartments 120 are etched into a silicon substrate, filled with a drug to be delivered, and sealed with a thin metal/dielectric layer or cap, as represented by anode 110. Each reservoir 120 is directly connected to an electrode, i.e., anode 110, which is used to electrically break the seal layer by applying a low voltage between cathode 105 and anode 110 and, thus, releasing the encapsulated drug.

FIG. 2 illustrates a cross-sectional view of an implantable glucose sensor device 200 based on catalytic oxidation of glucose forming peroxide and subsequent anodic dissociation of peroxide. In this exemplary device 200, a thin cap or barrier 210 covers a reservoir comprising a glucose sensor 310. As known in the art, glucose oxidase gel 220 is used as the sensor material. When a voltage is applied to electrodes 105 and 110, the current passing between the electrodes, breaks the thin cap 210 and the glucose oxidase is exposed. The reservoir 120 is preferably filled with isotonic fluid or a gel material. In a preferred embodiment the cap can be directly attached to the glucose oxidase 220. Determination of the glucose level, based on the glucose oxidase is well-known and need not be discussed in detail herein. See for example U.S. patent application Ser. No. 10/980,551, published May 5, 2005 as USPPA 2005/0096587.

If a voltage is applied between the two electrodes 311 and 312, a current can be measured corresponding to the glucose level. In a preferred embodiment the cap 210, is a thin freestanding film comprising a sandwich or a bi-layer of a polymer film and a very thin metal film. This composite is deposited in a way that it has a pre-strain, which improves opening or releasing behavior of the compartments.

FIG. 3 illustrates an exemplary control device 300 for controlling the activation of an array of sensors, similar to that shown in FIG. 1, using a passive matrix technology. In this case, compartments 120 are arranged to form an array of compartments and each compartment 120 includes at least one sensor 310. In one aspect the plurality of compartments 120 may be arranged in row and columns, wherein each row and each column can be individually attached to a voltage source. In the illustrated matrix, the row electrodes are connected to a select driver 320, which can switch between a first and second voltage (e.g., 0 and −0.5 Volts). The column electrodes are connected to the release or opening driver 330. In this exemplary configuration, to open or release a compartment, the row electrode associated with the row of compartments incorporating the desired compartment is switched to a second voltage while all other row voltages are maintained at the first voltage and the voltage in the column electrode associated with the desired compartment is set to an opening voltage, e.g., +0.5 Volts. In this case the one volt (1V) difference is sufficient to activate the associated release mechanism and open the desired compartment.

FIG. 4 illustrates an exemplary control device 400 for controlling an array of sensors, similar to that shown in FIG. 1, using active matrix technology. In this case, an active device or component 420, shown as a transistor, is associated with each compartment to active the release of a compartment. More specifically, to open a desired compartment, the active devices in the row containing the desired compartment are switched into a conducting state by applying a positive voltage to the illustrated transistor gate electrode 425. A voltage in the column containing the desired compartment is also set to the opening voltage (e.g., 1 Volt) and applied to a first terminal 427 of active device 420. The opening voltage is passed through the conducting active device to the electrode associated with the compartment. All other voltages are set to a zero value. Although not shown, it would be understood that the second electrode is set to reference voltage (e.g., 0 Volts) and the applied opening voltage is measured between the compartment electrodes. In other aspects of the invention, the compartment release mechanism may be facilitated by a resistive heating of the compartment seal 210. In this case, the device may incorporate an internal current source at each compartment. Operation of such control devices and also the control device shown in FIG. 4 is more fully discussed in commonly-owned European Patent Application Serial No. EP05106081.2, filed Jul. 5, 2005, the contents of which are incorporated by reference, and need not be discussed in detail herein.

FIG. 5 illustrates an exemplary control device 500 for controlling and sensing an array of sensors, similar to that shown in FIG. 1, in accordance with the principles of the invention. In this exemplary embodiment, active matrix technology, as discussed with regard to FIG. 4, is used to open a desired compartment to expose the associated sensor as previously described, i.e., application of an opening voltage on the appropriate column and a turn-on voltage on the appropriate row. In addition, each sensor 310 is attached to a second active device or component 510 that is switched to an “on” or conducting state when a desired compartment is opened and the sensor is exposed. With the second active device in a conducting state, measurements obtained by sensor 310, as represented by a voltage or current, are routed through second active device 510 and provided to a corresponding sensing line 515. The sensing line is connected to sensing driver 520.

In this exemplary embodiment, both addressing and activation of individual caps and sensing may be performed using only a single active matrix drive device.

FIG. 6A illustrates an exemplary embodiment of a local amplification circuit wherein sensor 310 generates a current signal (Iₛₑₙₛₑ) that is applied to an operational amplifier circuit to locally amplify the current. In this illustrated embodiment, the sensor current, Iₛₑₙₛₑ, is amplified by the value of the feedback resistor, R. Although FIG. 6A illustrates one type of operational amplifier, it would be known that operational amplifiers containing from one to up to several tens of transistors may be used and can be realized in large area electronics based upon low temperature poly-Si (LTPS) technology.

FIG. 6B illustrates a second exemplary embodiment of a local amplification circuit wherein a sensor 310 generates a current signal (Iₛₑₙₛₑ) that is combined with an inverter based circuit used to locally amplify the sensor signal and generate an output voltage. More specifically, an initial voltage is applied to the point Vₛₑₙₛₑ at the input to the inverter. When the Vₛₑₙₛₑ signal is high, the output of the inverter is V1. At this point the sensor device begins to work and the sense current (Iₛₑₙₛₑ) discharges the capacitor towards V_{ref}. When the capacitor charging takes Vₛₑₙₛₑ to a sufficiently low voltage, the inverter will switch and the output becomes V2.

In this exemplary embodiment, the sense current may be used to determine the time before the output switches. The higher the current, the faster the switch occurs.

Although the present invention has been discussed with regard to low temperature poly-Si (LTPS) based active matrix device, it would be recognized that amorphous-Si thin film transistor (TFT), microcrystalline or nano-crystalline Si, high temperature poly SiTFT, other anorganic TFTs based upon e.g. CdSe, SnO or organic TFTs may be used and consisted within the scope of the invention. Similarly, MIM, i.e., metal-insulator-metal devices or diode devices, for example using the double diode with reset (D2R) active matrix addressing methods, may be used to develop the invention disclosed herein.

While there has been shown, described, and pointed out fundamental novel features of the present invention as applied to preferred embodiments thereof, it will be understood that various omissions and substitutions and changes in the apparatus described, in the form and details of the devices disclosed, and in their operation, may be made by those skilled in the art without departing from the spirit of the present invention.

It is expressly intended that all combinations of those elements that perform substantially the same function in substantially the same way to achieve the same results are within the scope of the invention. Substitutions of elements from one described embodiment to another are also fully intended and contemplated.

What is claimed is:

1. An apparatus for managing and monitoring a sensing device (310) encapsulated in a compartment (120) formed within a medium (100), said medium compartment (120) including a release mechanism (210) suitable for exposing the sensing device (310), the apparatus comprising:
an active component (510) connected to the encapsulated sensing device (310), said active component (510) providing a measurement from the sensing device (310) to a sensing measurement device (520).

2. The apparatus of claim 1, wherein the sensing device further comprises:
an amplifying circuit (610).

3. The apparatus of claim 1, wherein the sensing measurement device further comprises:
a voltage measurement circuit (620).

4. The apparatus of claim 1, wherein the sensing measurement device further comprises:
a current measurement circuit (620).

5. The apparatus of claim 1, wherein the sensing measurement device comprises:
a plurality of sensing devices.

6. The apparatus of claim 5, wherein the sensing measurement device further comprises:
an active matrix array of sensing devices.

7. The apparatus of claim 1, wherein the release mechanism is a one time release mechanism.

8. The apparatus of claim 1, wherein the release mechanism is a closure cap.

9. The apparatus of claim 1, wherein each sensor (310) is associated with at least one release mechanism.

10. The apparatus of claim 1, wherein the release mechanism is operated using a passive matrix drive means.

11. The apparatus of claim 1, further comprising
a second active component (420) connected to an electrode associated with the release mechanism, said second active component (420) selectively providing an electrical signal to the electrode (110) for activating the release mechanism (200) and exposing the encapsulated sensing device (310).

12. The apparatus of claim 11, wherein the active component is selected from the group consisting of: transistor, diode and MIM device.

13. The apparatus of claim 1, wherein the active component is selected from the group consisting of: transistor, diode and MIM device.

14. The apparatus of claim 11, wherein the release mechanism is operated using an active matrix driving method.

15. The apparatus of claim 11, wherein the second active component (420) associated with the release mechanism and the active component (510) associated with the sensing device are associated with the same active matrix entity.

16. The apparatus of claim 15, wherein application of the voltage to the first and second active component is substantially concurrent.

17. The apparatus of claim 11, wherein the active components (510, 420) are fabricated from material selected from the group consisting of: amorphous-Si, poly-Si, microcrystalline or nano-crystalline Si, other anorganic semiconductors (CdSe, SnO), organic semiconductors, hydrogenated amorphous Silicon nitride, and oxides of tantalum.

18. The apparatus of claim 1, wherein the release mechanism (210) comprises:
a polymer film; and
a thin metal film.

19. A bio-implantable device (500) comprising:
a plurality of electrically exposable compartments (120), each of the compartments containing a release mechanism (200) encapsulating a sensor (310) therein;
an active component (510) connected to the encapsulated sensing device (310), said active component (510) providing a measurement from the sensing device (310) to a sensing measurement device (520).

20. The device of claim 19, wherein the plurality of compartments are arranged in an array.

21. The device of claim 20, wherein the sensing measurement device further comprises:
an array of sensing devices associated with an active matrix array.

22. The device of claim 19, further comprising:
a second active component (420) electrically connected to an associated one of the plurality of compartments (120) for providing a voltage to operate the release mechanism (210) and expose the encapsulated sensor (310) of a selected compartment (110).

23. The device of claim 22, wherein the release mechanism is operated using an active matrix driving method.

24. The apparatus of claim 23, wherein the second active component (420) associated with the release mechanism and the active component (510) associated with the sensing device are associated with the same active matrix entity.

25. The device of claim 19, wherein the sensing device, further comprises:
an amplifying circuit (610).

26. The device of claim 19, wherein the sensing device further comprises:
a voltage measurement circuit (620).

27. The device of claim 19, wherein the release mechanism (200) comprises:
a polymer film; and
a thin metal film.

28. The device of claim 19, wherein the sensing device further comprises:
a current measurement circuit (620).
